(19) United States
(12) Patent Application Publication
(10) Pub. No.: US 2006/0106354 A1
(43) Pub. Date: May 18, 2006

(54) Ostomy bag

(76) Inventor: Jacquelin Vantroostenberghe, Holland, TX (US)

Correspondence Address: ROBERT GREESON;7th Floor, 900 Washington Avenue, P.O. Box 1470, Waco, TX, 77603-1470, US

(21) Appl. No.: 10/990,758

(22) Filed: Nov. 17, 2004

(51) Int. Cl. A61F 5/44

(52) U.S. Cl. 604/335; 604/334

(57) ABSTRACT

An improved ostomy bag having a vent combination that allows for the easy and convenient expulsion of excess gas without the mess of emptying the entire bag of liquid waste. The valve combination also allows hygienic and convenient flushing and cleansing of the bag without the insertion of an irrigation bottle into the bottom discharge portion of the bag. A connection port attached to the bottom discharge portion of the bag provides for the easy attachment of a tube to allow the liquid waste to be directed to a sanitary and convenient waste receptacle rather than simply splashing out into a basin or sink.

### BACKGROUND OF THE INVENTION

1. Field of the Invention

The present invention relates to the field of medical equipment, and more specifically the field of ostomy bags.

2. Background Information

Each year approximately 100,000 people of all ages have a surgical procedure which results in the wearing of some sort of ostomy appliance. An ostomy is a man-made stoma, or opening, which is created in the abdominal wall through which waste material passes out of the body from the bowel. For the convenience and hygiene of the patient, various ostomy appliances or devices are used to collect this waste and to dispose of it in as hygienic and socially acceptable a manner as possible. An example of the current state of the art of these ostomy appliances can be seen in U.S. Pat. No. 6,419,664, which is incorporated herein by reference.

The creation of an ostomy is required as a result of many varied conditions and diseases including cancer, trauma, malformations present from birth, obstructions, complications from diverticulosis, Crohn's disease, ulcerative colitis, or familial polyposis. When it is the colon that is affected by one of these conditions or diseases, a colostomy is performed and the patient ends up with either a colostomy bag or pad, depending on the area of the colon involved. When the ileum is involved, which is the last portion of the small intestine, an ileostomy bag is frequently required. It is these ostomy and ileostomy bags that present several difficulties to the patient that are addressed by the current invention. Problems specifically associated with an ileostomy bag will be further discussed; however, it should be understood that similar problems arise with other types of ostomy bags. Applicant's invention is thought to also be of particular benefit to patients needing these other types of ostomy bags (namely colostomy bags) as well.

In a healthy gastrointestinal tract, the nutrients in food are primarily digested and absorbed in the small intestine and the leftover waste leaves the small intestine in an essentially liquid form. Next the excess liquid is absorbed in the colon, creating solid stool which passes out of the body voluntarily through the rectum. Because an ileostomy results in a stoma created from the ileum, which as previously noted is part of the small intestine, the waste that is involuntarily and automatically excreted from the stoma is essentially liquid. In addition, a certain quantity of gas is naturally produced in the ileum and must pass through the stoma into the ileostomy bag. This liquid waste and gas must be cleared from the ileostomy bag in a manner that is as convenient and sanitary as possible in order to provide the patient with a higher quality of life. The procedure which is currently used to clear an ileostomy bag is inconvenient at best and is usually performed in an unhygienic manner. The present inventor has worked in the field and has observed this procedure on numerous occasions.

The procedure which is currently employed is potentially unsanitary and requires the health professional to don not only gloves, but gown and mask. Because of factors such as patient positioning and lack of ability or desire to cooperate, the waste product often splashes during the emptying and cleansing process and endangers the health professional. The apparatus and method that comprise the system of the present invention solve the problems faced by a typical ileostomy bag wearer and his/her care giver.

The schedule followed by a typical ileostomy bag wearer is to change his/her bag every two or three days and to empty and irrigate the bag as needed between changes. Currently, an ileostomy bag has one opening down at the bottom which drains stool and the accompanying liquid and which is used to irrigate the bag between changes. The only way to release any buildup of gas in the bag is to unclip the one bottom opening, drain any material in the bag, and release the gas. This process is cumbersome and messy to say the least.

The typical process for emptying under the current system is to unclip the bottom opening in the bag and to drain the contents into a container of some sort. The container, unfortunately, is typically a bathroom sink for an individual at home. The bag wearer simply cannot empty the bag into a toilet or more appropriate receptacle because this would require stooping or kneeling at the edge of the toilet. Most bag wearers are not physically able to do this, or are not able to do this and manipulate the bag at the same time without spilling or splashing liquid stool on the toilet or floor.

In a hospital setting, the nurse typically drains the bag's contents into a basin. This basin must, of course, be transported to the appropriate disposal area. This procedure has great potential for splashes and spills. In addition, the opportunity for exposure to germs is great.

After draining, the bag wearer or care-giver typically takes an irrigation bottle and inserts the tip into the bottom opening of the bag—unfortunately where stool is still clinging—and squirts water into the bag, letting the water drain back out into the sink, or, in the case of nurse or care giver, the basin. As can well be imagined, either way this is a messy and unhygienic process. Once the bag is cleared of the liquid stool and gas, the bottom opening is reclipped. This process, of course, leaves a sink that needs to be cleaned and disinfected, or a basin to be transported and cleaned, along with a contaminated irrigation bottle.

With the improvements provided by the current invention, the bag wearer or attendant can empty and rinse the bag directly into a toilet or disposable bag and prevent much exposure to germs and bacteria.

In addition to the unhygienic procedure of draining the liquid stool that is dictated by the current state of ileostomy bag technology, there is the additional problem of a lack of an easy, convenient way to release any build up of gas in the bag. The current method is so unsanitary and messy that ileostomy bag wearers are frequently embarrassed to go out in public because of gas build-up. When gas builds up in the bag, it distends and protrudes the bag, showing through most clothing. In addition, the build-up of gas jeopardizes the integrity of the seal around the stoma and can cause embarrassing leaks. The bag wearer is typically set up at home to deal with this problem, however unhygienically, but out in public or when visiting others, it becomes a real challenge that frequently results in the bag wearer staying home. The current invention addresses this problem also.

### SUMMARY OF THE INVENTION

The general purpose of the present invention, which will be described subsequently in greater detail, is to provide an improved ostomy bag which has many of the advantages of such bags known in the art and many novel features that result in a new ostomy bag which is not anticipated, rendered obvious, suggested, or even implied by any of the known ostomy bags, either alone or in any combination thereof.

In view of the foregoing, it is an object of the present invention to provide a more hygienic and convenient ostomy bag.

It is another object of the present invention to provide the ostomy bag wearer with a more suitable, hygienic, and convenient method with which to clean his/her bag between changings.

It is another object of the present invention to provide the ostomy bag wearer with a hygienic, convenient manner with which to expel excess gas from the bag in order to prevent embarrassment in public.

It is a further object of the present invention to provide an improved ostomy bag which, through the use of a tubing connection, tube, and top vent, will allow the wearer to conveniently, easily, and hygienically empty and cleanse his/her bag between changings.

It is another object of the present invention to provide an improved ostomy bag which, through the use of a top vent, will allow the wearer to easily, conveniently, and discreetly expel excess gas from his/her bag between cleanings.

In satisfaction of these and other related objectives, Applicant's present invention provides an improved ostomy bag which, through the use of a conveniently placed top vent, allows gas expulsion without resorting to a complete draining and cleansing of the bag. In addition, the use of the top vent along with a tube and tube connection placed in the bottom opening of the ostomy bag, allows the wearer to hygienically and easily empty, flush, and cleanse his/her bag without messy spills or the use of the bathroom or kitchen sink.

Applicant's improved ostomy bag permits a wearer to function more naturally in public by allowing for easy gas expulsion, and facilitates the cleansing process of the bag by providing for a more hygienic method of emptying and cleaning the bag without the use of a sink, allowing the waste to be easily emptied directly into a toilet.

### BRIEF DESCRIPTION OF THE DRAWINGS

FIG. 1 is a plane view of a preferred embodiment of the present invention.

### DETAILED DESCRIPTION OF THE PREFERRED EMBODIMENT

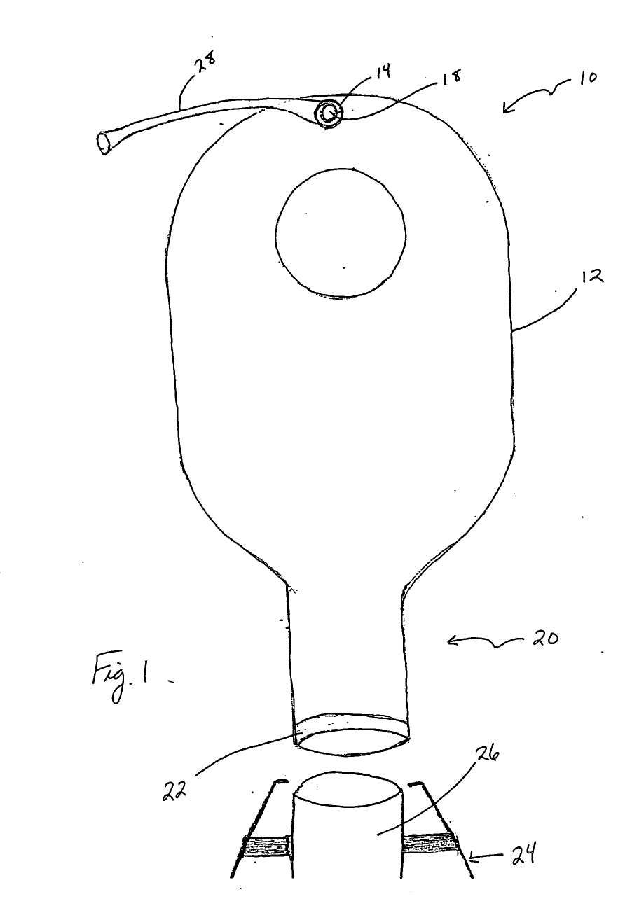

With reference to FIG. 1, the improved collecting bag of the present invention is identified generally by the reference number 10.

Improved collecting bag 10 includes a conventional ostomy bag 12 (as can be seen in U.S. Pat. No. 6,419,664 B1, which is incorporated herein by reference) with venting port 18, which allows for the venting of excess gas build-up without requiring the bag wearer to completely undo and empty ostomy bag 12, as is commonly done now.

In the preferred embodiment, shown in FIG. 1, venting port 18 is fitted with a one-way valve 14, such as is commonly used in beach blow-up toys or other inflatable items. The bag wearer need simply to compress the bag manually to allow the gas to expel from collecting bag 10. That is, as sufficient pressure builds within the bag, valve 14 is actuated so as to allow gas to seep there through. This allows the wearer to conveniently and discretely rid collecting bag 10 of embarrassing gas build-up. Without one-way valve 14, the gas could escape continuously or at any time from collecting bag 10. This could lead to embarrassing social situations. Other useful embodiments are envisioned where valve 14 allows gas to flow from the bag as a bag wearer or attendant manually actuates the valve. In such embodiment, valve 14 is actuated by applying pressure about the valve. Upon actuation, valve 14 allows fluid to seep there through.

Referring again to FIG. 1, collecting bag 10 has an elongate discharge portion 20 which is long enough to accommodate a connection port 22. In the preferred embodiment, connection port 22 is substantially rigid and is comprised of plastic or other similar material. Connection port 22 is attached to elongate discharge portion 20 in conventional manner, either by glue, bonding, or other means. Elongate discharge portion 20 is also long enough to fold over to seal using a closeable clip. This sealing method is well known in the medical field When folded over and clipped shut, connection port 22 lies flat against collecting bag 10 and against the bag wearer giving minimal discomfort.

When elongate discharge portion 20 is unclipped and folded open, a tube 26 of appropriate width and length can be inserted through connection port 22 and into elongate discharge portion 20, and then secured with a spring clip 24 such as is used commonly in the medical field. This configuration allows the liquid contents of collecting bag 10 to empty conveniently into a toilet, disposable bag, or other suitable receptacle. Typically what is done now is that the ostomy bag 12 is emptied into the sink so that the bag wearer does not have to bend or kneel beside the toilet. The method described here allows the bag wearer to sit on a toilet, fit the tube into his/her bag and empty the contents hygienically down into the bowl of the toilet. Or, in the case of a health professional helping the bag wearer, the health professional can empty the collecting bag 10 directly into a disposable container without fear of contamination through splashing. This improvement prevents the spread of germs caused by splashing and emptying fecal matter into a sink.

After emptying the contents of the improved collecting bag 10, the bag wearer can hygienically and conveniently rinse and cleanse the bag by inserting a syringe such as is commonly used in the field, not shown, filled with flushing liquid, such as plain water or water with a disinfectant such as is commonly used in the field and not shown, and forcing such flushing liquid into and through the one-way valve 14 and into collecting bag 10. In the preferred embodiment, the excess flushing liquid flows through collecting bag 10, out of tube 26, and into the toilet or other convenient and hygienic disposal receptacle.

Referring to FIG. 1, in this embodiment tail 28 is attached by conventional means to collecting bag 10 and is in direct contact with port 18 and one-way valve 14. Discharge stem 28 is made of a flexible tubing typically known in the field or other similar material. Discharge stem 28 aides the bag wearer in the manipulation of one-way valve 14 and venting port 18. Without such a discharge stem 28, the bag wearer could easily dislodge the ostomy bag 12 from his or her body through the pulling motions that would be required to vent excess gas from collecting bag 10 or to irrigate collecting bag 10 through the use of flushing liquid.

Although the invention has been described with reference to specific embodiments, this description is not meant to be construed in a limited sense. Various modifications of the disclosed embodiments, as well as alternative embodiments of the inventions will become apparent to persons skilled in the art upon reference to the description of the invention. It is, therefore, contemplated that the appended claims will cover such modifications that fall within the scope of the invention including such modifications as pertain to an ostomy bag in addition to the modifications to an ileostomy bag as disclosed herein.

I claim:

1. A discharge fluid transport device, comprising:
a fluid holding member having a first aperture, a second aperture, and a third aperture;
where said first aperture is configured to receive discharge fluid and allow said fluid to flow into said fluid holding member;
where said second aperture is in combination with a valve member, where said valve member may be actuated to allow for the displacement of fluid from said fluid holding member;
where said third aperture is configured to reversibly engage with a waste receptacle and is further configured to allow fluid to flow therethrough.

2. The device of claim 1, where
said valve member is further in combination with a stem member configured to allow the directional discharge of said fluid from said fluid holding member.

3. An improved device for directing discharge fluid, comprising:
a valve member in combination with a fluid holding member, where said valve member may be actuated to allow for the displacement of said discharge fluid from said fluid holding member;
a stem member extending from said valve member and having a proximate portion and a distal portion, where said discharge fluid flows through said stem member, and where said stem member may be manipulated to allow for directional discharge of said fluid.

4. An improved method for disposing of discharge fluid, comprising:
selecting a discharge fluid transport device, comprising:
a fluid holding member having a first aperture, a second aperture, and a third aperture;
where said first aperture is configured to receive discharge fluid and allow said fluid to flow into said fluid holding member;
where said second aperture is in combination with a valve member, where said valve member may be actuated to allow for the displacement of fluid from said fluid holding member;
where said third aperture is configured to reversibly engage with a waste receptacle and is further configured to allow fluid to flow therethrough.
flowing a cleansing fluid through said valve member, through said fluid holding member, and though said third aperture until substantially all of said discharge fluid has been carried away from said fluid holding member.

5. An improved method for disposing of discharge fluid, comprising:
selecting a discharge fluid transport device, comprising:
a fluid holding member having a first aperture, a second aperture, and a third aperture;
where said first aperture is configured to receive discharge fluid and allow said fluid to flow into said fluid holding member;
where said second aperture is in combination with a valve member, where said valve member may be actuated to allow for the displacement of fluid from said fluid holding member;
where said third aperture is configured to reversibly engage with a waste receptacle and is further configured to allow fluid to flow therethrough;
where said valve member is further in combination with a stem member configured to allow the directional discharge of said fluid from said fluid holding member;
flowing a cleansing fluid through said valve member, through said fluid holding member, and though said third aperture until substantially all of said discharge fluid has been carried away from said fluid holding member.
